(19) United States
(12) Patent Application Publication
(10) Pub. No.: US 2006/0222755 A1
(43) Pub. Date: Oct. 5, 2006

(54) System and method for loading a beneficial agent into holes in a medical device

(75) Inventors: Stephen Hunter Diaz, Palo Alto, CA (US); Theodore L. Parker, Danville, CA (US)

Correspondence Address: CINDY A. LYNCH;CONOR MEDSYSTEMS, INC., 1003 HAMILTON COURT, MENLO PARK, CA, 94025, US

(73) Assignee: Conor Medsystems, Inc., Menlo Park, CA (US)

(21) Appl. No.: 11/392,318

(22) Filed: Mar. 28, 2006

Related U.S. Application Data
(60) Provisional application No. 60/667,733, filed on Mar. 31, 2005.

(51) Int. Cl. A61F 2/06; B05D 3/00

(52) U.S. Cl. 427/2.21; 623/1.42

(57) ABSTRACT

A system for loading a beneficial agent into holes in a medical device includes a punch system for punching plugs of beneficial agent from a thin sheet into the holes in the medical device. The loading of a beneficial agent in the form of a thin film allows the formation of multilayered structures within the holes to control release kinetics and prevent any meniscus which occurs when a beneficial agent is deposited as a liquid in the holes and dried. The punch type loading system also can provide the ability to deliver large and potentially sensitive molecules including proteins, enzymes, antibodies, antisense, ribozymes, gene/vector constructs, and cells including endothelial cells.

### CROSS REFERENCE TO RELATED APPLICATIONS

This application claims priority to U.S. Provisional Patent Application Ser. No. 60/667,733, filed Mar. 31, 2005, the entire contents of which are incorporated here by reference.

### FIELD OF THE INVENTION

The invention relates to a method and apparatus for loading a beneficial agent, such as a drug into holes in a medical device, such as a stent, by punching a thin film of beneficial agent.

### DESCRIPTION OF THE RELATED ART

Implantable medical devices are often used for delivery of a beneficial agent, such as a drug, to an organ or tissue in the body at a controlled delivery rate over an extended period of time. These devices may deliver agents to a wide variety of bodily systems to provide a wide variety of treatments.

One of the many implantable medical devices which have been used for local delivery of beneficial agents is the coronary stent. Coronary stents are typically introduced percutaneously, and transported transluminally until positioned at a desired location. These devices are then expanded either mechanically, such as by the expansion of a mandrel or balloon positioned inside the device, or expand themselves by releasing stored energy upon actuation within the body. Once expanded within the lumen, these devices, called stents, become encapsulated within the body tissue and remain a permanent implant.

Known stent designs include monofilament wire coil stents (U.S. Pat. No. 4,969,458); welded metal cages (U.S. Pat. Nos. 4,733,665 and 4,776,337); and, most prominently, thin-walled metal cylinders with axial slots formed around the circumference (U.S. Pat. Nos. 4,733,665; 4,739,762; and 4,776,337). Known construction materials for use in stents include polymers, organic fabrics and biocompatible metals, such as stainless steel, gold, silver, tantalum, titanium, and shape memory alloys, such as Nitinol, and biodegradable materials including biodegradable polymers and biodegradable metal alloys.

Of the many problems that may be addressed through stent-based local delivery of beneficial agents, one of the most important is restenosis. Restenosis is a major complication that can arise following vascular interventions such as angioplasty and the implantation of stents. Simply defined, restenosis is a wound healing process that reduces the vessel lumen diameter by extracellular matrix deposition, neointimal hyperplasia, and vascular smooth muscle cell proliferation, and which may ultimately result in renarrowing or even reocclusion of the lumen. Despite the introduction of improved surgical techniques, devices, and pharmaceutical agents, the overall restenosis rate for bare metal stents is still reported in the range of 10% to 25% within six to twelve months after an angioplasty procedure. To treat this condition, additional revascularization procedures are frequently required, thereby increasing trauma and risk to the patient.

One of the techniques recently introduced to address the problem of restenosis is the use of surface coatings of various drugs on stents. Surface coatings, however, can provide little actual control over the release kinetics of beneficial agents. These coatings are necessarily very thin, typically 5 to 8 microns deep. The surface area of the stent, by comparison is very large, so that the entire volume of the beneficial agent has a very short diffusion path to discharge into the surrounding tissue.

Increasing the thickness of the surface coating has the beneficial effects of improving drug release kinetics including the ability to control drug release and to allow increased drug loading. However, the increased coating thickness results in increased overall thickness of the stent wall and increased risk of cracking, flaking, or separating from the stent.

In addition, it is not currently possible to deliver many drugs with a surface coating due to sensitivity of the drugs to water, other compounds, or conditions in the body which degrade the drugs. Lack of drug capacity and lack of control over delivery also limit the usefulness of surface coatings for many drugs.

U.S. Patent Publication 2004/0073294 describes systems and methods for loading a beneficial agent into holes in a medical device, such as a stent. This process uses a computer guided micro dispenser to load droplets of liquid solution into the holes of the stent. The stents are mounted on a rubber coated mandrel blocking the bottoms of the holes. A machine, using machine vision, maps the exact locations of each of the target holes and then moves each hole under the dispenser that then loads liquid into the holes. The filled stent is dried in an oven, and then a next deposit is applied. Subsequent deposits of polymer and polymer/drug are applied to achieve the desired release properties.

This process has some advantages. It is a non-contact process, so there is little drag of material from hole to hole and no back contamination. It is very fast, filling at least 10 holes per second. The dispenser can be turned on and off very quickly, so complex patterns of filling can be supported. It has proven results of accuracy and consistency.

The liquid droplet method also has some limitations. The piezoelectric dispenser generally requires solutions with low viscosities. Therefore, the solids content should remain low, often less than 5%. The low solids content can result in the need for many deposits to build up a sufficient amount of beneficial agent. In addition, the solid should be very soluble in the solvent. This may require the use of solvents that have undesirable properties. Finally, the oven drying step is too hot for some drugs or sensitive proteins.

Accordingly, it would be desirable to provide a system and method for loading a beneficial agent into an expandable medical device, such as a stent, which can deliver compositions with higher solids content and/or can operate with limited drying time or low drying temperature.

It would also be desirable to provide a system and method for loading beneficial agents such as agents with little or no shelf life into a medical device just prior to use of the medical device.

### SUMMARY OF THE INVENTION

The present invention relates to a system and method for loading a beneficial agent into holes in a medical device wherein the beneficial agent is in the form of a thin film which is loaded by punching.

In accordance with one aspect of the invention, a method for loading a medical device with a beneficial agent comprises the steps of providing a medical device with an exterior surface and a plurality of holes intersecting the exterior surface, providing a film of a beneficial agent and pressing plugs of the film into the holes in the medical device.

In accordance with a further aspect of the invention, a system for loading a medical device with a beneficial agent is comprised of a holder for supporting a medical device having a plurality of holes for receiving a beneficial agent, a film of a beneficial agent, and at least one punch configured to press plugs of the film into the holes in the medical device.

### BRIEF DESCRIPTION OF THE DRAWINGS

The invention will now be described in greater detail with reference to the preferred embodiments illustrated in the accompanying drawings, in which like elements bear like reference numerals.

FIG. 1 is a schematic perspective view of a punch system for loading a beneficial agent into a medical device.

### DETAILED DESCRIPTION OF THE INVENTION

The present invention relates to a method and apparatus for loading a beneficial agent into holes in a medical device. More particularly, the invention relates to a method and apparatus for loading a beneficial agent into holes in a stent by punching a thin film of the beneficial agent into the holes.

First, the following terms, as used herein, shall have the following meanings:

The term “beneficial agent” as used herein is intended to have its broadest possible interpretation and is used to include any therapeutic agent or drug, as well as inactive agents such as barrier layers, carrier layers, therapeutic layers or protective layers.

The terms “drug” and “therapeutic agent” are used interchangeably to refer to any therapeutically active substance that is delivered to a living being to produce a desired, usually beneficial, effect. The present invention is particularly well suited for the delivery of antineoplastic, angiogenic factors, immuno-suppressants, anti-inflammatories and antiproliferatives (anti-restenosis agents) such as paclitaxel and Rapamycin for example, and antithrombins such as heparin, for example. The present invention is also well suited for delivery of larger and potentially sensitive molecules including proteins and stem cells.

The term “matrix” or “biocompatible matrix” are used interchangeably to refer to a medium or material that, upon implantation in a subject, does not elicit a detrimental response sufficient to result in the rejection of the matrix. The matrix typically does not provide any therapeutic responses itself, though the matrix may contain or surround a therapeutic agent, a therapeutic agent, an activating agent or a deactivating agent, as defined herein. A matrix is also a medium that may simply provide support, structural integrity or structural barriers. The matrix may be polymeric, non-polymeric, hydrophobic, hydrophilic, lipophilic, amphiphilic, and the like.

The term “bioresorbable” refers to a matrix, as defined herein, that can be broken down by either chemical or physical process, upon interaction with a physiological environment. The bioresorbable matrix is broken into components that are metabolizable or excretable, over a period of time from minutes to years, preferably less than one year, while maintaining any requisite structural integrity in that same time period.

The term “polymer” refers to molecules formed from the chemical union of two or more repeating units, called monomers. Accordingly, included within the term “polymer” may be, for example, dimers, trimers and oligomers. The polymer may be synthetic, naturally-occurring or semisynthetic. In preferred form, the term “polymer” refers to molecules which typically have a M_{w} greater than about 3000 and preferably greater than about 10,000 and a M_{w} that is less than about 10 million, preferably less than about a million and more preferably less than about 200,000.

The term “holes” refers to holes of any shape and includes both through-holes and recesses.

Implantable Medical Devices with Holes

U.S. Pat. No. 6,241,762 illustrates a medical device in the form of a stent designed with large, non-deforming struts, which can contain holes without compromising the mechanical properties of the struts, or the device as a whole. The non-deforming struts can be achieved by the use of ductile hinges which are described in detail in U.S. Pat. No. 6,241,762, which is incorporated hereby by reference in its entirety. The holes serve as large, protected reservoirs for delivering various beneficial agents to the device implantation site. The stent described above or any other known stent can be provided with holes for delivery of beneficial agents according to the present invention.

The holes can be circular, oval, rectangular, polygonal, D-shaped, or other shaped and can extend through the thickness of the medical device. The volume of beneficial agent that can be delivered using holes is about 3 to 10 times greater than the volume of a 5 micron coating covering a stent with the same stent/vessel wall coverage ratio. This much larger beneficial agent capacity provides several advantages. The larger capacity can be used to deliver multi-drug combinations, each with independent release profiles, for improved efficacy. Also, larger capacity can be used to provide larger quantities of less aggressive drugs to achieve clinical efficacy without the undesirable side-effects of more potent drugs.

According to one example, the total depth of the holes is about 100 to about 140 microns (about 0.0039 to about 0.0055 inches), typically 125 microns (0.0049 inches) for stainless steel. For stronger alloys, such as commercially available cobalt chromium alloys, the stent may be somewhat thinner. For example, the total depth of the holes is about 60 to about 100 microns (about 0.0026 to about 0.0039 inches) for cobalt chromium alloys. According to one preferred embodiment of the present invention, each of the holes have an area of at least 5×10⁻⁶ square inches, and preferably at least 10×10⁻⁶ square inches. A square hole having a width of about 0.005 inches will have an hole area of about 25×10⁻⁶ square inches.

Uses for Implantable Medical Devices

Although the present invention has been described with reference to a medical device in the form of a stent, the medical devices of the present invention can also be medical devices of other shapes useful for site-specific and time-release delivery of drugs to the body including the heart and other organs and tissues. The drugs may be delivered to the vasculature including the coronary and peripheral vessels for a variety of therapies, and to other lumens in the body. The drugs may increase lumen diameter, create occlusions, or deliver the drug for other reasons. The medical devices can take a variety of shapes including cylinders, spheres, coils, filament, mesh, and other shapes.

Medical devices and stents, as described herein, are useful for the prevention of amelioration of restenosis, particularly after percutaneous transluminal coronary angioplasty and intraluminal stent placement. In addition to the timed or sustained release of anti-restenosis agents, other agents such as anti-inflammatory agents and immunosuppressant agents may be incorporated into the microstructures incorporated in the plurality of holes within the device. This allows for site-specific treatment or prevention of any complications routinely associated with stent placements that are known to occur at very specific times after the placement occurs.

A size and number of the holes will depend on the particular medical device, beneficial agent, and treatment desired. For example, the width of the holes can vary from about 0.001 inches to about 0.1 inches, preferably about 0.001 inches to about 0.05 inches.

Systems and Methods for Loading a Beneficial Agent into a Medical Device

A system for loading a beneficial agent into holes in a medical device includes a punch system for punching plugs of beneficial agent from a thin sheet into the holes in the medical device. The loading of a beneficial agent in the form of a thin film allows the formation of multilayered structures within the holes to control release kinetics and prevent any meniscus which occurs when a beneficial agent is deposited as a liquid in the holes and dried. The punch type loading system also can provide the ability to deliver large and potentially sensitive molecules including proteins, enzymes, antibodies, antisense, ribozymes, gene/vector constructs, and cells including endothelial cells.

A multilayer sheet of beneficial agent can be fabricated by a variety of methods to include layers of drug, drug/polymer, polymer, or other matrix material. The multilayer sheet can be formed with layers with different compositions or different concentrations of the same beneficial agents in the layers. Different layers can be comprised of different therapeutic agents altogether, creating the ability to release different therapeutic agents at different points in time. The layers of beneficial agent provide the ability to tailor a drug delivery profile to different applications. This allows the medical device according to the present invention to be used for delivery of a variety of beneficial agents to a wide variety of locations in the body.

In one example, the multilayer sheet is fabricated by the spin coating methods known for use in applying photoresist in the chip manufacturing industry. Spin coating provides the ability to produce large multilayer sheets with accurate thickness of the film controllable by adjusting the properties and temperature of the materials. The spin coating process can be used to form a layered structure with polymer blocking layers on the outer surfaces to control release rate or release direction. Intermediate polymer layers can be used to create pulsatile or multi-stage release profiles. The use of different solvents for the different layers in the layered structure of the film can create a film with distinct layers. When solvents that dissolve an underlying layer are used, a film with indistinct layers and concentration gradients of drug in the film can be created.

Other methods for creating the multilayer sheet of beneficial agent include coextrusion or coating methods, such as dip, spray, curtain, or roll coating. Although the invention has been described with reference to a multilayer film sheet which is punched into the holes, in some cases a homogeneous film or a film with a concentration gradient, but without distinct layers can be used.

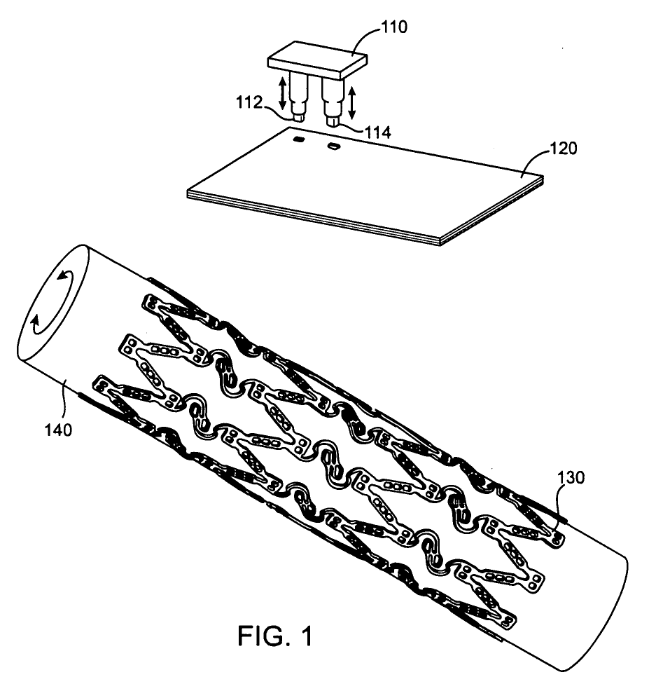

The system for loading the medical device or stent with the beneficial agent film shown in FIG. 1 includes a punching apparatus 110 for punching the film 120 directly into the holes in the stent 130. According to one embodiment of the method, the stents are mounted on a mandrel 140 or other holder and mapped in the manner described in U.S. Patent Publication 2004/0073294, which is incorporated herein by reference, to determine the precise location of each of the holes. The multilayer sheet is then positioned over the stent and a computer controlled punching system is used to punch a plug out of the sheet 120 into each of the holes while moving the punch and/or the stent to align the punch with the holes. The punch may include a single punch which is moved to each hole in the stent or a series of punches, such as a row of punches corresponding to a stent struts. In the event that the holes are of multiple shapes or sizes, multiple punches 112, 114 should be provided. After the holes are loaded with the punch, the whole stent can be exposed to solvent vapors or solvent in a liquid form to glue the plugs firmly into the holes by swelling and softening the exterior layers of the plug and thus, bonding the plug to the holes.

According to this embodiment, the punching of the plugs is performed by using the edges of the hole as a die. However, because the size and shape of the holes are somewhat variable and because of the rounded top and bottom edges of the hole, the punch will have a relatively large clearance. This requires that the beneficial agent film is fabricated to be somewhat brittle to allow the plug to break out even thought the punch and die are not a tight match. This brittle beneficial agent film would not be suitable for use as a coating on a medical device.

According to another alternative, the punching system will create plugs from the beneficial agent sheet which stored in a holder and then placed from the holder into the holes in the stent. In this system, the punch and die would have close tolerances eliminating the need to use brittle beneficial agent sheets and allow the use of tougher materials. The punched plugs can be stacked in a holder tube and dispensed and pressed into the holes by computer controlled ejection from the holder tube. The ejection system can include an air jet.

Another alternative embodiment for transporting the punched disks to the stent holes is to place the disks onto a pressure sensitive tape. The punched plugs are then pressed into the holes in a manner similar to a typewriter, as each disk is positioned over the hole, the punch would drive the disk into the hole. The bond between the disk and the tape is weak enough for the disk to lift off the tape when it is pressed into the hole.

In one embodiment, different holes can be filled with different agents by providing two or more films containing different agents which are placed into holes in the stent, such as alternating holes, or different agents on the ends and center of the stent.

Other therapeutic agents for use with the present invention may, for example, take the form of small molecules, peptides, lipoproteins, polypeptides, polynucleotides encoding polypeptides, lipids, protein-drugs, protein conjugate drugs, enzymes, oligonucleotides and their derivatives, ribozymes, other genetic material, cells, antisense oligonucleotides, monoclonal antibodies, platelets, prions, viruses, bacteria, eukaryotic cells such as endothelial cells, stem cells, ACE inhibitors, monocyte/macrophages and vascular smooth muscle cells. Such agents can be used alone or in various combinations with one another. For instance, anti-inflammatories may be used in combination with antiproliferatives to mitigate the reaction of tissue to the antiproliferative. The therapeutic agent may also be a pro-drug, which metabolizes into the desired drug when administered to a host. In addition, therapeutic agents may be pre-formulated as microcapsules, microspheres, microbubbles, liposomes, niosomes, emulsions, dispersions or the like before they are incorporated into the matrix. Therapeutic agents may also be radioactive isotopes or agents activated by some other form of energy such as light or ultrasonic energy, or by other circulating molecules that can be systemically administered.

Exemplary classes of therapeutic agents include antiproliferatives, antithrombins (i.e., thrombolytics), immunosuppressants, antilipid agents, anti-inflammatory agents, antineoplastics including antimetabolites, antiplatelets, angiogenic agents, anti-angiogenic agents, vitamins, antimitotics, metalloproteinase inhibitors, NO donors, nitric oxide release stimulators, anti-sclerosing agents, vasoactive agents, endothelial growth factors, beta blockers, AZ blockers, hormones, statins, insulin growth factors, antioxidants, membrane stabilizing agents, calcium antagonists (i.e., calcium channel antagonists), retinoids, anti-macrophage substances, antilymphocytes, cyclooxygenase inhibitors, immunomodulatory agents, angiotensin converting enzyme (ACE) inhibitors, anti-leukocytes, high-density lipoproteins (HDL) and derivatives, cell sensitizers to insulin, prostaglandins and derivatives, anti-TNF compounds, hypertension drugs, protein kinases, antisense oligonucleotides, cardio protectants, petidose inhibitors (increase blycolitic metabolism), endothelin receptor agonists, interleukin-6 antagonists, anti-restenotics, vasodilators, and other miscellaneous compounds.

Antiproliferatives include, without limitation, paclitaxel, actinomycin D, rapamycin, everolimus, ZoMaxx, tacrolimus, cyclosporin, and pimecrolimus.

Antithrombins include, without limitation, heparin, aspirin, sulfinpyrazone, ticlopidine, ABCIXIMAB, eptifibatide, tirofiban HCL, coumarines, plasminogen, [symbol] ₂-antiplasmin, streptokinase, urokinase, bivalirudin, tissue plasminogen activator (t-PA), hirudins, hirulogs, argatroban, hydroxychloroquin, BL-3459, pyridinolcarbamate, Angiomax, and dipyridamole.

Immunosuppressants include, without limitation, cyclosporine, rapamycin and tacrolimus (FK-506), ZoMaxx, everolimus, etoposide, and mitoxantrone.

Antilipid agents include, without limitation, HMG CoA reductase inhibitors, nicotinic acid, probucol, and fibric acid derivatives (e.g., clofibrate, gemfibrozil, gemfibrozil, fenofibrate, ciprofibrate, and bezafibrate).

Anti-inflammatory agents include, without limitation, pimecrolimus, salicylic acid derivatives (e.g., aspirin, insulin, sodium salicylate, choline magnesium trisalicylate, salsalate, dflunisal, salicylsalicylic acid, sulfasalazine, and olsalazine), para-amino phenol derivatives (e.g., acetaminophen), indole and indene acetic acids (e.g., indomethacin, sulindac, and etodolac), heteroaryl acetic acids (e.g., tolmetin, diclofenac, and ketorolac), arylpropionic acids (e.g., ibuprofen, naproxen, flurbiprofen, ketoprofen, fenoprofen, and oxaprozin), anthranilic acids (e.g., mefenamic acid and meclofenamic acid), enolic acids (e.g., piroxicam, tenoxicam, phenylbutazone and oxyphenthatrazone), alkanones (e.g., nabumetone), glucocorticoids (e.g., dexamethaxone, prednisolone, and triamcinolone), pirfenidone, and tranilast.

Antineoplastics include, without limitation, nitrogen mustards (e.g., mechlorethamine, cyclophosphamide, ifosfamide, melphalan, and chlorambucil), methylnitrosoureas (e.g., streptozocin), 2-chloroethylnitrosoureas (e.g., carmustine, lomustine, semustine, and chlorozotocin), alkanesulfonic acids (e.g., busulfan), ethylenimines and methylmelamines (e.g., triethylenemelamine, thiotepa and altretamine), triazines (e.g., dacarbazine), folic acid analogs (e.g., methotrexate), pyrimidine analogs (5-fluorouracil, 5-fluorodeoxyuridine, 5-fluorodeoxyuridine monophosphate, cytosine arabinoside, 5-azacytidine, and 2′,2′-difluorodeoxycytidine), purine analogs (e.g., mercaptopurine, thioguanine, azathioprine, adenosine, pentostatin, cladribine, and erythrohydroxynonyladenine), antimitotic drugs (e.g., vinblastine, vincristine, vindesine, vinorelbine, paclitaxel, docetaxel, epipodophyllotoxins, dactinomycin, daunorubicin, doxorubicin, idarubicin, epirubicin, mitoxantrone, bleomycins, plicamycin and mitomycin), phenoxodiol, etoposide, and platinum coordination complexes (e.g., cisplatin and carboplatin).

Antiplatelets include, without limitation, insulin, dipyridamole, tirofiban, eptifibatide, abciximab, and ticlopidine.

Angiogenic agents include, without limitation, phospholipids, ceramides, cerebrosides, neutral lipids, triglycerides, diglycerides, monoglycerides lecithin, sphingosides, angiotensin fragments, nicotine, pyruvate thiolesters, glycerol-pyruvate esters, dihydoxyacetone-pyruvate esters and monobutyrin.

Anti-angiogenic agents include, without limitation, endostatin, angiostatin, fumagillin and ovalicin.

Vitamins include, without limitation, water-soluble vitamins (e.g., thiamin, nicotinic acid, pyridoxine, and ascorbic acid) and fat-soluble vitamins (e.g., retinal, retinoic acid, retinaldehyde, phytonadione, menaqinone, menadione, and alpha tocopherol).

Antimitotics include, without limitation, vinblastine, vincristine, vindesine, vinorelbine, paclitaxel, docetaxel, epipodophyllotoxins, dactinomycin, daunorubicin, doxorubicin, idarubicin, epirubicin, mitoxantrone, bleomycins, plicamycin and mitomycin.

Metalloproteinase inhibitors include, without limitation, TIMP-1, TIMP-2, TIMP-3, and SmaPI.

NO donors include, without limitation, L-arginine, amyl nitrite, glyceryl trinitrate, sodium nitroprusside, molsidomine, diazeniumdiolates, S-nitrosothiols, and mesoionic oxatriazole derivatives.

NO release stimulators include, without limitation, adenosine.

Anti-sclerosing agents include, without limitation, collagenases and halofuginone.

Vasoactive agents include, without limitation, nitric oxide, adenosine, nitroglycerine, sodium nitroprusside, hydralazine, phentolamine, methoxamine, metaraminol, ephedrine, trapadil, dipyridamole, vasoactive intestinal polypeptides (VIP), arginine, and vasopressin.

Endothelial growth factors include, without limitation, VEGF (Vascular Endothelial Growth Factor) including VEGF-121 and VEG-165, FGF (Fibroblast Growth Factor) including FGF-1 and FGF-2, HGF (Hepatocyte Growth Factor), and Ang1 (Angiopoietin 1).

Beta blockers include, without limitation, propranolol, nadolol, timolol, pindolol, labetalol, metoprolol, atenolol, esmolol, and acebutolol.

Hormones include, without limitation, progestin, insulin, the estrogens and estradiols (e.g., estradiol, estradiol valerate, estradiol cypionate, ethinyl estradiol, mestranol, quinestrol, estrond, estrone sulfate, and equilin).

Statins include, without limitation, mevastatin, lovastatin, simvastatin, pravastatin, atorvastatin, and fluvastatin.

Insulin growth factors include, without limitation, IGF-1 and IGF-2.

Antioxidants include, without limitation, vitamin A, carotenoids and vitamin E.

Membrane stabilizing agents include, without limitation, certain beta blockers such as propranolol, acebutolol, labetalol, oxprenolol, pindolol and alprenolol.

Calcium antagonists include, without limitation, amlodipine, bepridil, diltiazem, felodipine, isradipine, nicardipine, nifedipine, nimodipine and verapamil.

Retinoids include, without limitation, all-trans-retinol, all-trans-14-hydroxyretroretinol, all-trans-retinaldehyde, all-trans-retinoic acid, all-trans-3,4-didehydroretinoic acid, 9-cis-retinoic acid, 11-cis-retinal, 13-cis-retinal, and 13-cis-retinoic acid.

Anti-macrophage substances include, without limitation, NO donors.

Anti-leukocytes include, without limitation, 2-CdA, IL-1 inhibitors, anti-CD116/CD18 monoclonal antibodies, monoclonal antibodies to VCAM, monoclonal antibodies to ICAM, and zinc protoporphyrin.

Cyclooxygenase inhibitors include, without limitation, Cox-1 inhibitors and Cox-2 inhibitors (e.g., CELEBREX® and VIOXX®).

Immunomodulatory agents include, without limitation, immunosuppressants (see above) and immunostimulants (e.g., levamisole, isoprinosine, Interferon alpha, and Interleukin-2).

ACE inhibitors include, without limitation, benazepril, captopril, enalapril, fosinopril sodium, lisinopril, quinapril, ramipril, spirapril, and 2B3 ACE inhibitors.

Cell sensitizers to insulin include, without limitation, glitazones, P PAR agonists and metformin.

Antisense oligonucleotides include, without limitation, resten-NG.

Cardio protectants include, without limitation, VIP, pituitary adenylate cyclase-activating peptide (PACAP), apoA-I milano, amlodipine, nicorandil, cilostaxone, and thienopyridine.

Petidose inhibitors include, without limitation, onmipatrilat.

Anti-restenotics include, without limitation, include vincristine, vinblastine, actinomycin, epothilone, paclitaxel, paclitaxel derivatives (e.g., docetaxel), rapamycin, rapamycin derivatives, everolimus, tacrolimus, ZoMaxx, and pimecrolimus.

PPAR gamma agonists include, without limitation, farglitizar, rosiglitazone, muraglitazar, pioglitazone, troglitazone, and balaglitazone.

Miscellaneous compounds include, without limitation, Adiponectin.

Agents may also be delivered using a gene therapy-based approach in combination with an expandable medical device. Gene therapy refers to the delivery of exogenous genes to a cell or tissue, thereby causing target cells to express the exogenous gene product. Genes are typically delivered by either mechanical or vector-mediated methods.

Some of the agents described herein may be combined with additives which preserve their activity. For example additives including surfactants, antacids, antioxidants, and detergents may be used to minimize denaturation and aggregation of a protein drug. Anionic, cationic, or nonionic detergents may be used. Examples of nonionic additives include but are not limited to sugars including sorbitol, sucrose, trehalose; dextrans including dextran, carboxy methyl (CM) dextran, diethylamino ethyl (DEAE) dextran; sugar derivatives including D-glucosaminic acid, and D-glucose diethyl mercaptal; synthetic polyethers including polyethylene glycol (PEF and PEO) and polyvinyl pyrrolidone (PVP); carboxylic acids including D-lactic acid, glycolic acid, and propionic acid; detergents with affinity for hydrophobic interfaces including n-dodecyl- [symbol] -D-maltoside, n-octyl- [symbol] -glucoside, PEO-fatty acid esters (e.g. stearate (myrj 59) or oleate), PEO-sorbitan-fatty acid esters (e.g. Tween 80, PEO-20 sorbitan monooleate), sorbitan-fatty acid esters (e.g. SPAN 60, sorbitan monostearate), PEO-glyceryl-fatty acid esters; glyceryl fatty acid esters (e.g. glyceryl monostearate), PEO-hydrocarbon-ethers (e.g. PEO-10 oleyl ether; triton X-100; and Lubrol. Examples of ionic detergents include but are not limited to fatty acid salts including calcium stearate, magnesium stearate, and zinc stearate; phospholipids including lecithin and phosphatidyl choline; CM-PEG; cholic acid; sodium dodecyl sulfate (SDS); docusate (AOT); and taumocholic acid.

While the invention has been described in detail with reference to the preferred embodiments thereof, it will be apparent to one skilled in the art that various changes and modifications can be made and equivalents employed, without departing from the present invention.

What is claimed is:

1. A method for loading a medical device with a beneficial agent, the method comprising:
providing a medical device with an exterior surface and a plurality of holes intersecting the exterior surface;
providing a film of a beneficial agent; and
pressing plugs of the film into the holes in the medical device.

2. The method of claim 1, wherein beneficial agent includes a drug and a carrier.

3. The method of claim 2, wherein the carrier is a polymer.

4. The method of claim 1, wherein the method creates a medical device with substantially no beneficial agent on a surface of the medical device outside of the holes.

5. The method of claim 1, further comprising securing the plugs of film in the holes by liquefying a portion of the plugs.

6. The method of claim 5, wherein the portion of the plugs are liquefied with a solvent vapor.

7. The method of claim 1, wherein the plurality of holes of the medical device and the plurality of fixture holes have a width of about 0.001 inches to about 0.1 inches.

8. The method of claim 5, wherein portion of the plugs are liquefied by heating.

9. The method of claim 1, wherein the beneficial agent includes a solvent.

10. The method of claim 1, wherein the film of beneficial agent is a multilayer sheet.

11. The method of claim 1, wherein the plurality of holes in the medical device are through holes.

12. The method of claim 1, wherein the medical device is a coronary stent.

13. The method of claim 1, wherein the beneficial agent is a protein.

14. A system for loading a medical device with a beneficial agent, the system comprising:
a holder for supporting a medical device having a plurality of holes for receiving a beneficial agent;
a film of a beneficial agent; and
at least one punch configured to press plugs of the film into the holes in the medical device.

15. The system of claim 14, wherein the beneficial agent includes a drug and a carrier.

16. The system of claim 14, wherein the medical device is a stent.
